(19) United States
(12) Patent Application Publication
(10) Pub. No.: US 2023/0035741 A1
(43) Pub. Date: Feb. 2, 2023

(54) TRANSCUTANEOUS GASEOUS MEASUREMENT DEVICE

(71) Applicant: Wocester Polytechnic Institute, Worcester, MA (US)

(72) Inventors: Ulkuhan Guler, Shrewsbury, MA (US); Tuna B. Tufan, Worcester, MA (US)

(21) Appl. No.: 17/960,105

(22) Filed: Oct. 4, 2022

Related U.S. Application Data
(63) Continuation-in-part of application No. 17/066,570, filed on Oct. 9, 2020.
(60) Provisional application No. 63/252,251, filed on Oct. 5, 2021.
(60) Provisional application No. 62/913,299, filed on Oct. 10, 2019.

(51) Int. Cl. A61B 5/1455; A61B 5/00

(52) U.S. Cl. CPC A61B 5/14556; A61B 5/6833; A61B 5/7246

(57) ABSTRACT

A transdermal patch measures a gaseous concentration based on transcutaneous diffusion through an epidermal surface of a patient. The patch employs an indicator responsive to a gaseous presence for emitting light having an intensity and lifetime (duration) based on the gaseous presence. An optical receptor is in communication with logic for receiving the intensity of emitted light and computing a gaseous concentration based on the received intensity and lifetime (duration). A wireless transmitter conveys the results to a base station or monitoring counterpart for untethered patient monitoring. Low power demands and circuit footprint are amenable to a wearable device such as a patch for continuous use.

### RELATED APPLICATIONS

This patent application claims the benefit under 35 U.S.C. § 119(e) of U.S. Provisional Patent App. No. 63/252,251, filed Oct. 5, 2021, entitled “TRANSCUTANEOUS GASEOUS MONITOR DEVICE,” and is a Continuation-in-Part (CIP) under 35 U.S.C. § 120 of U.S. patent application Ser. No. 17/066,570, filed Oct. 9, 2020, entitled “WEARABLE BLOOD GAS MONITOR,” which claims the benefit under 35 U.S.C. § 119(e) of U.S. Provisional Patent App. No. 62/913,299, filed Oct. 10, 2019, entitled “WEARABLE BLOOD GAS MONITOR,” all incorporated herein by reference in entirety.

### BACKGROUND

Blood gas testing is an effective tool for analysis and diagnosis of many human physiological parameters. Blood chemistry can be an effective indicator of many bodily functions and conditions. Among the vital signs of human body, respiration is a key component of a person's health. Respiratory health can be quantified by rate, volume, and blood-gas content. Traditional respiration monitoring methods such as arterial blood gas monitoring and pulse oximetry have certain advantages and disadvantages.

### SUMMARY

A transdermal patch measures a gaseous concentration based on transcutaneous diffusion through an epidermal surface of a patient. Transcutaneous oxygen and carbon dioxide differ from a measurement of saturated gases often measured in a patient blood flow or tissue. The patch employs an indicator responsive to an oxygen presence for emitting light having an intensity and lifetime based on the gaseous presence. An optical receptor is in communication with logic for receiving the intensity and lifetime (i.e. duration) of emitted light and computing the oxygen concentration based on the received intensity and lifetime. Low power demands and circuit footprint are amenable to a wearable device such as a patch for continuous use.

Configurations herein are based, in part, on the observation that oxygen sensing is frequently employed in many physiologic contexts due to its prevalent nature in essential human respiration. Oxygen in various forms and concentrations is found abundantly throughout living tissue, and therefore is often a reliable indicator of proper and healthy physiology. Unfortunately, conventional approaches to oxygen monitoring rely on oxygen saturation in blood, relating to hemoglobin-bound oxygen (oxyhemoglobin) in oxygenated blood. Measurement of oxygen concentration in tissue conventionally requires a robust apparatus with heating and power requirements inconsistent with a portable device. Oxygen concentration measures the oxygen concentration based on partial pressure of oxygen dissolved in the bloodstream, and is often preferred to a measurement of oxygen saturation, or may be taken in conjunction with saturated O₂.

Accordingly, configurations herein substantially overcome the shortcomings of conventional, bulky oxygen detection by providing a wearable oxygen sensor in the form of a patch or epidermal appliance for measuring transcutaneous oxygen upon diffusion through the epidermal surface. A photoluminescent indicator emits light responsive to an illuminating stimuli in a manner that varies with the presence of the diffused oxygen. An electronic circuit performs computations and implements logic for determining the oxygen concentration of the underlying tissue based on the partial pressure of the oxygen computed by the system.

In further detail, configurations below disclose a system and method implemented by a blood gas measurement device, including an optical source operable for emitting light, a sensing film adapted for adherence to an epidermal surface and responsive to gaseous diffusion from the epidermal surface. The sensing film has a luminophore responsive to emit light responsive to the optical source, such that the emitted light is based on a gaseous diffusion through the light sensitive medium. A photodetector sensitive to re-emitted light from the sensing film is coupled to an electronic circuit having logic responsive to a signal from the photodetector for computing a level of a blood gas based on the re-emitted light. A wireless transmitter completes the wearable device for monitoring patient oxygen concentration in a form factor suitable for implementation as a small patch or similar untethered personal device.

In a particular configuration directed to oxygen in the blood, the optical source emits light in a blue spectrum, and the photodetector is sensitive to light in a red spectrum. The sensing film may be a luminescent sensing film adapted for adherence to an epidermal surface. In one configuration, the luminescence of the fluorophore functional groups is quenched in the presence of oxygen, reducing the intensity and lifetime of the re-emitted red light. Change in the intensity and lifetime of the re-emitted red light received by the photodetector is inversely proportional to the change in the concentration of oxygen based on the partial pressure of transcutaneous oxygen (PtcO₂) diffusing from the epidermal surface. The wavelength sensitivity is specific to particular luminophores in the luminescent sensing film, as is the remitted light. Alternative sensing films may involve different wavelengths. For example, a rubidium based sensing film is responsive to a greener color light and remits a more orange wavelength. Various sensing films may be employed, and the optical sources and photodetectors matched to the wavelength sensitivity for producing a response to oxygen. Still further, a sensitivity to other gases may also be employed.

In contrast to conventional approaches, the PtcO₂ sensor and associated logic is encapsulated in a wearable patch or low-profile, adhesive arrangement and includes a wireless transmitter for offloading storage and analytics capability. While conventional SpO₂ measurement has been achieved in finger-attached wired sensors, compact sensing of PtcO₂ oxygen levels has typically required a larger footprint device including a heating element for electrochemical sensing of perfusion. Therefore, conventional approaches employ an electrochemical, rather than optical measurement medium and are not feasible for miniaturization due to the power demands of the heating element.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a system context diagram including a wearable sensor patch suitable for use with configurations herein;

FIG. 2 shows a side cutaway view of the wearable patch of FIG. 1 in communication with an epidermal surface of a patient;

FIG. 3 shows a schematic view of a light intensity and processing circuit for the wearable patch of FIG. 2;

FIG. 4 is a diagram of light emission energy received by the circuit of FIG. 3;

FIG. 5 is a graph of the light wavelengths emitted in the diagram of FIG. 4;

FIG. 6 is a block diagram of the wearable patch as described in FIGS. 3-5;

FIGS. 7A-7D show graphs of voltage and light pulses in the circuit of FIG. 6;

FIGS. 8A-8C shows a further detail of the analog front end of FIG. 6;

FIG. 9 shows a relation between intensity and lifetime (duration) of remitted light;

FIG. 10 shows a context diagram of an alternate configuration for gaseous monitoring and reporting;

FIG. 11 shows a schematic diagram of the configuration of FIG. 10 optimized for carbon dioxide detection;

FIG. 12 shows a block diagram of the device in FIG. 10; and

FIGS. 13A and 13B show results of the device of FIGS. 10-12 for measuring transcutaneous carbon dioxide.

### DETAILED DESCRIPTION

The description below presents an example of a wearable device for measurement of oxygen (O₂) by transcutaneous partial pressure (PtcO₂), which differs from conventional measurement because conventional approaches measure saturated oxygen (SpO₂). Saturated oxygen is bound to hemoglobin, while PtcO₂ measurement refers to a concentration of total oxygen. Depending on the medical context, PtcO₂ based readings have an advantage over conventional SpO₂ either alone or in conjunction with SpO₂ readings.

A blood gas measurement device includes an optical source and a sensing film adapted for adherence to an epidermal surface and responsive to gaseous diffusion from the epidermal surface. The sensing film is sensitive to the gas or gases targeted for sensing based on re-emittance properties that are affected by transdermal gaseous diffusion. An optical source, typically an LED for low power and heat properties, emits a light directed to the sensing film. A photodetector is sensitive to re-emitted light from the sensing film based on the optical source, and logic responsive to a signal from the photodetector computes a level of a blood gas based on the re-emitted light.

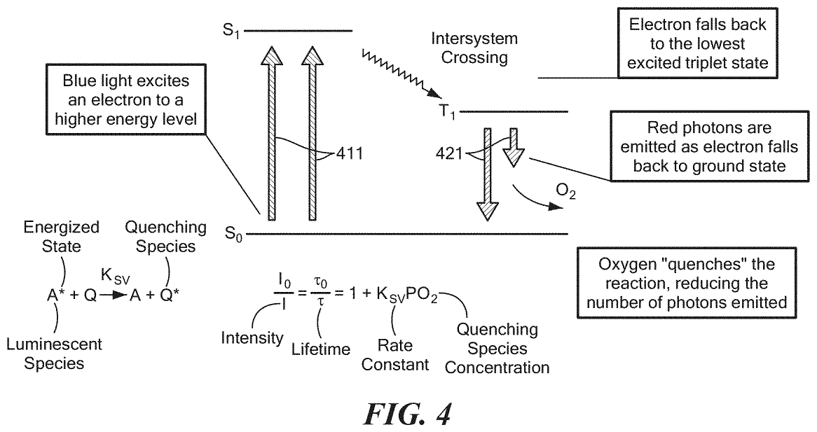

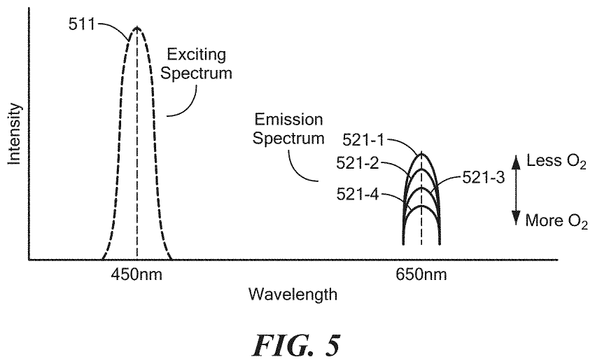

Photoluminescence refers to the emission of photons produced in certain molecules during de-excitation and is one of the possible physical effects resulting from the interaction between light and matter. When a luminescent molecule absorbs a photon, it is excited from a ground state to some higher vibrational level, and emits light upon its return to the lower state. The subsequent de-excitation processes are depicted below in FIGS. 4 and 5.

In the presence of molecular oxygen, the photoluminescence of such molecules is quenched via a radiationless deactivation process which involves molecular interaction between the quencher and the luminophore (collisional quenching) and it is therefore diffusion limited. The mechanism by which oxygen quenches luminescence is not germane to the disclosed approach, however it has been suggested that the paramagnetic oxygen causes the luminophore to undergo intersystem crossing to the triplet state while molecular oxygen goes to the excited state) and then returns to ground state.

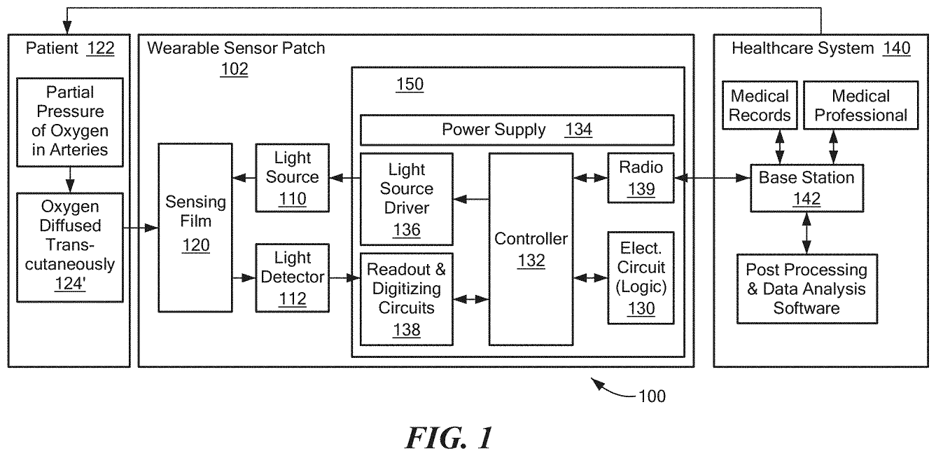

FIG. 1 is a system context diagram including a wearable sensor patch suitable for use with configurations herein. Referring to FIG. 1, a blood gas measurement device 100 includes an optical source 110 and a light sensitive medium 120 such as a sensing film. The light sensitive medium 120 is configured to emit light responsive to the optical source 110, in which the emitted light is based on a gaseous diffusion through the light sensitive medium 120. A photoreceptor 112 is disposed for receiving the emitted light, and logic 130 coupled to the receptor 112 computes a quantity of the gaseous diffusion based on the emitted light.

In an example configuration, the device 100 takes the form of a wearable patch 102 adhered to or adjacent to the skin (epidermal surface) of a patient 122 for disposing a sensing circuit 150 thereon. An electronic circuit and/or processor instruction sequence implements the logic 130 which computes the oxygen concentration based on the duration and intensity of the emitted light. The photoreceptor 112 receives the light, and readout and digitizing circuits 138 transform and digitize the received light into intensity and lifetime (duration) values employed by the logic 130. A controller 132 is driven by a power supply 134 for powering the readout and digitizing electronics 138, controller 132, radio 139 and a light source driver 136 activating the LED light source 110. The controller 132 couples to the readout and digitizing circuit 138 for receiving emitted light. A radio 139 wirelessly couples to a base station 142 for gathering and transporting the computed oxygen levels, and may take the form of a personal device, hospital monitor, data logger or any suitable network device for capturing the computed oxygen levels and related data without encumbering the patient 122 with bulky devices. The patient data may then be recorded, stored and analyzed according to the patient's healthcare regimen. In contrast to conventional tethered approaches which require an electronic connection (wire), the radio 139 implements a wireless connection to a monitoring base station 142 for receiving and coalescing patient data and generating needed alerts and reports resulting from the oxygen concentration.

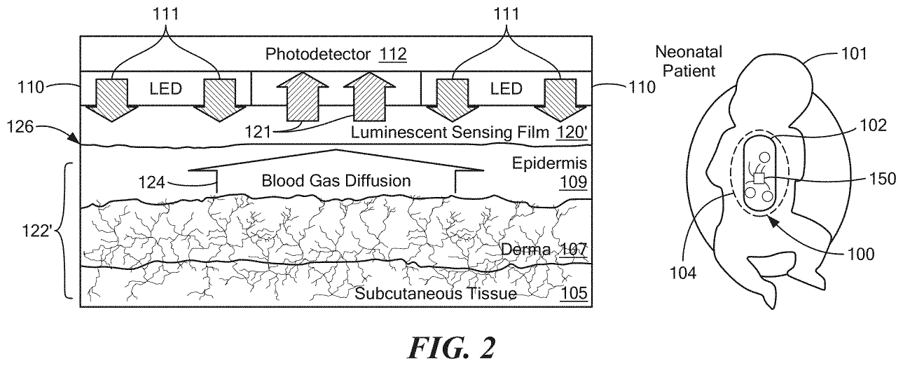

FIG. 2 shows a side cutaway view of the wearable patch 102 of FIG. 1 adhered to an epidermal portion 104 in communication with an epidermal surface 126 of the patient 122. Referring to FIGS. 1 and 2, the light sensitive medium 120 takes the form of a luminescent sensing film 120′ adapted for communication with a gaseous diffusion source for receiving the gaseous diffusion. Diffused gases 124 including oxygen diffuses through dermal layers 122′ of the patient 122 and through the sensing film 120. Subcutaneous tissue 105, derma 107 and epidermis 109 define the dermal layers 122′ that are the source of the transcutaneously diffused oxygen 124′. The sensing film 120 is maintained in communication with the epidermal surface 126 by a collection medium adapted to engage a surface for coupling the light sensitive medium to a source of the gaseous diffusion. The collection medium may take the form of an adhesive, taped, or strapped patch for directing the diffused gases 124 from the surface to the collection medium.

For example, in a particular use case, collection medium is a planar epidermal patch 102 adapted for receiving gaseous diffusion from an infant 101 patient. The ability to remotely monitor infants could improve the feasibility of early discharge and reduce the risk of undiagnosed issues becoming significant after hospital release. Continuous and accurate remote tracking of vital respiratory parameters in a fully wireless manner could provide relevant and accurate data to the caregiver to inform the course of treatment. Configurations herein address the need to monitor patience's transcutaneous oxygen level remotely and safely by a medical professional with a light and low-height profile wearable device. Conventional vital monitoring systems, especially those that monitor blood gas status are typically large, bulky bed-side machines with wired electrodes and are usually used in a hospital setting. These machines require the patient to be tethered to a hospital bed with limited mobility.

Dermal placement of the patch 102 can be made elsewhere such as the abdomen or torso where it is less susceptible to patient movement, further enhanced by the omission of wired tethers. Such a patch encloses the light sensitive medium 120 in a sealing engagement with the dermal surface 126 for quantifying the transdermal oxygen emitted or diffused through the patch.

It its most basic form, the patch 102 and sensing device 100 perform a method for sensing an oxygen concentration based on transcutaneous oxygen 124′ by receiving a diffusion of oxygen 124′ and other gases 124 through the transcutaneous surface 126. The patch 102 adheres the light sensitive medium 120 to the transcutaneous surface 126, such that the light sensitive medium 120 has a photoluminescent response to the diffused oxygen 124′. A pulsed light 111 from the light source 110 on the light sensitive medium 120 causes the photodetector 112 to receive an emitted light 121 in response to the diffused oxygen. The logic 130 computes the partial pressure of oxygen value based on an oxygen sensitive luminophore in the light sensitive medium 120 responsive to quenching of the emitted light 121 inversely with the oxygen presence. In other words, the emitted light “fades” faster with greater oxygen, now discussed in more detail below.

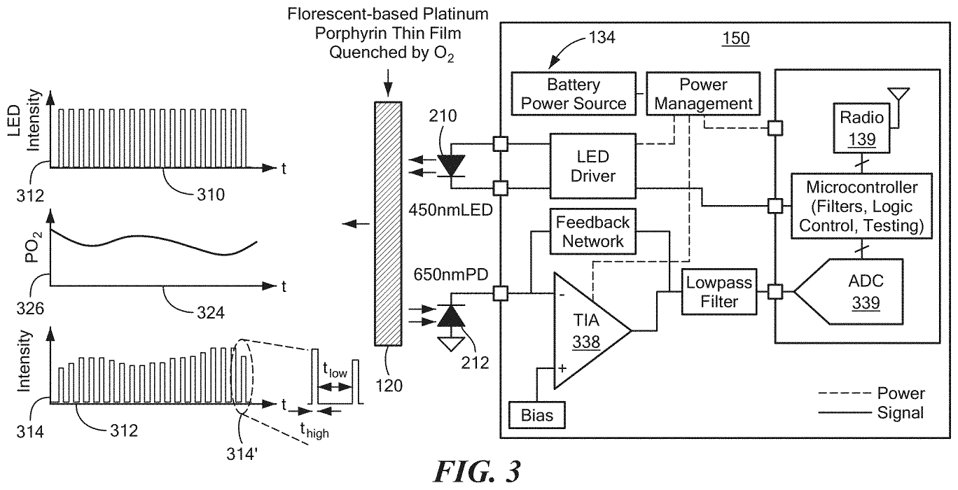

FIG. 3 shows a schematic view of a light intensity and processing circuit for the wearable patch of FIG. 2. Referring to FIGS. 1-3, as described above, the light sensitive medium 120 is a sensing film adapted for adherence to an epidermal surface 126 and responsive to gaseous diffusion from the epidermal surface. In FIG. 3, the device 100 is shown in a schematic of the fluorescent-based transcutaneous oxygen sensing system. A low power transimpedance amplifier and low power microcontroller connect to wireless system and powered by a coin cell battery. Alternate configurations may employ any suitable power arrangement, such as a coin cell, thin film battery, wireless power link, or similar methods. The disclosed system communicates to a base station 142 using a wireless protocol, such as Bluetooth®, Bluetooth LE, ZigBee®, WiFi, NFC (Near Field Communication) or similar approach consistent with FDA (Food and Drug Administration) and other governmental recommendations or guidelines for health related devices.

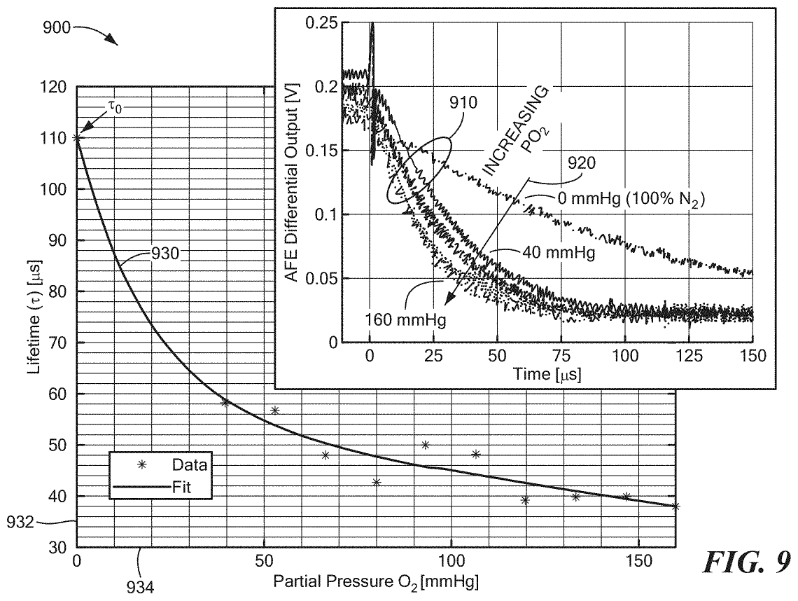

In the example of FIG. 3, the light source 110 is provided by a blue LED 210 having a wavelength around 450 nm. The LED 210 emits a pulsed intensity shown by graph 310 in a series of fixed intensity bursts or flashes. The light sensitive medium 120 is a luminescent sensing film of platinum porphyrin (Pt-porphyrin) or other luminescent material such as rubidium, and emits (or remits) a red light in a wavelength around 650 nm received by a red light photodetector 212. The luminescent sensing film, photoluminescent film, or simply thin film is a planar, sheet-like material having light emission properties as disclosed herein. The gaseous oxygen diffused 124′ during the blue light emission is shown in graph 324. It can be observed that an intensity 314 of the emitted red light varies inversely with the partial pressure (PO₂) 326 of diffused oxygen, responsive to constant intensity 312 pulses of blue light. The remitted red light varies based on a quenching effect of oxygen on an excitation of a luminophore in the light sensing medium 120, thus reducing the red light response as oxygen increases, as shown in dotted line region 314′ as the time t[high] transitions to t[low] in the presence of oxygen, described in further detail below. The luminescent material has the property of remitting light having an intensity and duration quenched by the presence of oxygen, therefore allowing oxygen measurement by observing the intensity and lifetime of the remitted light. The intensity/lifetime value of the remitted red light is received by a transimpedance amplifier 338 and converted by an analog/digital converter 339 for processing by logic 130. The intensity and lifetime are related as discussed below with respect to FIG. 9; in general, a lower oxygen presence mitigates the quenching effect and results in a greater intensity and lifetime of the remitted light. Measurement based on lifetime (duration) of the return pulses tends to be more resistant to factors such as LED fading/age and skin color variations.

FIG. 4 is a diagram of light emission energy received by the circuit of FIG. 3. Referring to FIGS. 2-4, in the excitation response of 314′, a quenching effect of oxygen on the photoluminescent sensing film 120′ is shown. The light sensitive medium 120 has a photoluminescent response and an oxygen based decay responsive the diffusion source for emitting a red light 121 inversely proportional with a partial pressure of oxygen in the gaseous diffusion 124. The sensing film 120′ includes an oxygen sensitive luminophore for exhibiting an emission of red light 121 in response to the blue light 111 based on a partial pressure of oxygen diffused through the light sensitive medium 120.

Benefits of the claimed approach will be apparent with reference to conventional approaches. Traditional devices measure PtcO₂ electrochemically, using methods requiring a heating element that increases the diffusion of O₂ from blood vessels, thus increasing the concentration of O₂ in the gas 124 above the targeted skin area. However, a heating element negatively affects the feasibility of a miniaturized PtcO₂ wearable as it substantially increases the wearable device size and the power requirement. In addition, the hotspot irritates and may even burn the skin during continuous monitoring.

To overcome such limitations, the disclosed approach employs a fluorescence-based method that allows the use of comfortable dry electrodes without the need for heating. This method uses the photoluminescent film including platinum porphyrin (Pt-porphyrin) or similar luminophore based medium. When a luminescent molecule absorbs a photon, it becomes excited from its ground state (S0) to some higher vibrational level of either the first or second electronic state (S1 or S2). When the film 120′ is exposed to blue light 411, it emits red light 421, the intensity and lifetime of which are inversely proportional to the concentration of O₂ 124 around the film as the energy level reaches S1 and following the excitation by the pulse of blue light, fall back to an energy level shown by S0. The fluorescence of the photoluminescent film is typically measured in terms of its lifetime (i.e. fall time) where t0 is the lifetime of the film fluorescence without the quencher (oxygen), and t is the lifetime of the fluorescence with the quencher. Conventional approaches refer to the so-called Stern-Volmer relationship in reference to the kinematics of quenching, discussed further below in FIG. 9.

The disclosed example includes a wearable or adhesive patch having the luminescent film through which patient-diffused oxygen passes. An oxygen presence passing through or adjacent the luminescent material causes the oxygen sensitive quenching response from the optical source. Various arrangements of luminescent materials in conjunction with the patient may be employed, along with corresponding photoreceptors and optical sources with light wavelengths (colors) based on the luminescent material. Similarly, targeted gases other than oxygen may be measured based on the luminescent film and gaseous sensitivity.

FIG. 5 is a graph of the light wavelengths emitted in the diagram of FIG. 4. Referring to FIGS. 3-5, the logic 130 is configured to compare the received red light 121 to a quenching effect of an oxygen concentration, such that the quenching effect increases with the oxygen concentration to indicate the partial pressure of oxygen in the diffused gases 124, and thus the oxygen levels in the blood and tissue underlying the patch 102. The light source 210 emits a blue light having an intensity 511, and the resulting excitation results in a red light remission having, for example an intensity of 521-1 when lower oxygen (PtcO₂), and increasing oxygen levels result in the red light intensity decreasing to levels of 521-2, 521-3, and to 521-4 as the increased oxygen quenches the red light response.

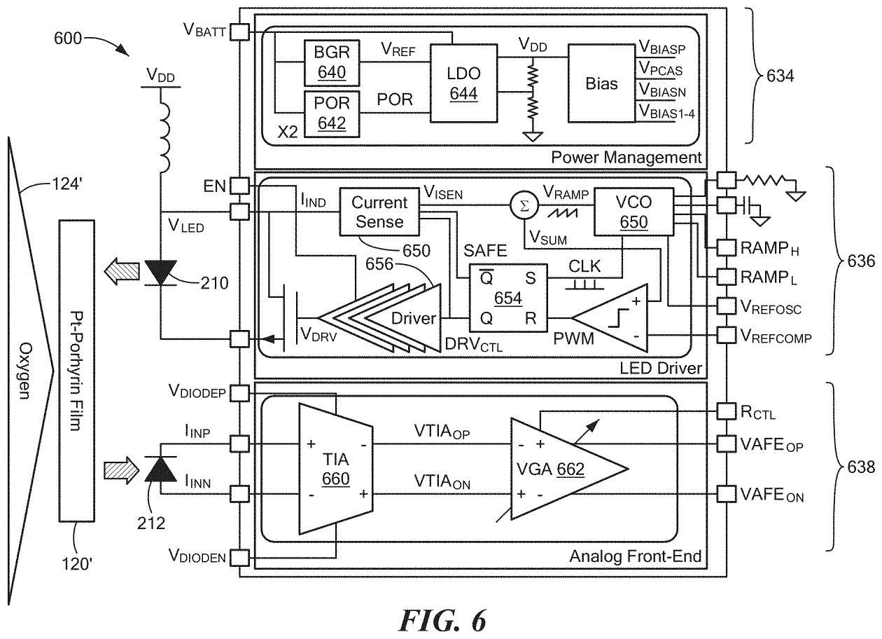

FIG. 6 is a block diagram of the wearable patch 102 as described in FIGS. 1-5, and FIGS. 7A-7D depict circuit schematics and graphs of the device 100. Referring to FIGS. 1-6 and 7A-7D, the circuit includes a power management portion 634 corresponding to the power supply 134, an LED driver 636 for powering and pulsing the blue LED 210, and an analog front end (AFE) 638 for receiving and generating a signal from the photodetector 212 corresponding to the remitted red light intensity indicative of the oxygen level. The logic 130 includes a mapping of luminescent diminution for an increasing oxygen availability, defined by a partial pressure of oxygen, that indicates the transcutaneous oxygen 124′ diffusing through the dermal (skin) surface 126. The quenching ability resulting from increasing oxygen 124′ causes the luminescent diminution, or intensity reduction, of the received red light 121 by the photodetector 212.

The power management portion 634 may be implemented as a Power Management Integrated Circuit (PMIC) including two bandgap references (BGR) 640, two power-on-reset (POR) blocks 642, two biasing circuits, and two low-dropout (LDO) regulators 644, powered off of an external 3 V battery. The BGR 640 includes 5 V CMOS devices to withstand a wide range of battery voltages (VBATT) and generates a stable reference voltage (VREF) of 1.2 V for the LDO, which converts the battery voltage to a stable 1.8 V supply voltage (VDD). A resistive feedback network sets the relationship between VREF and VDD. When the battery voltage drops below a certain level, the POR circuit provides a power-on reset signal for the LDO. The purpose of two LDO channels is to isolate the power path between the AFE and the LED driver and to distribute the load.

For example, the LED driver 136 excites the blue LED 210 with a peak wavelength of 450 nm, which excites the Pt-porphyrin film. The film emits red light of 650 nm, the intensity and lifetime of which are inversely proportional to the concentration of O₂. The current flowing through the photodetector 212 is proportional to the intensity of the red light from the film. Examples herein include Pt-porphyrin film having a sensitivity and responsiveness at the disclosed wavelengths. Alternate luminescent materials may of course be employed, such as rubidium based materials, and the wavelength values adjusted accordingly.

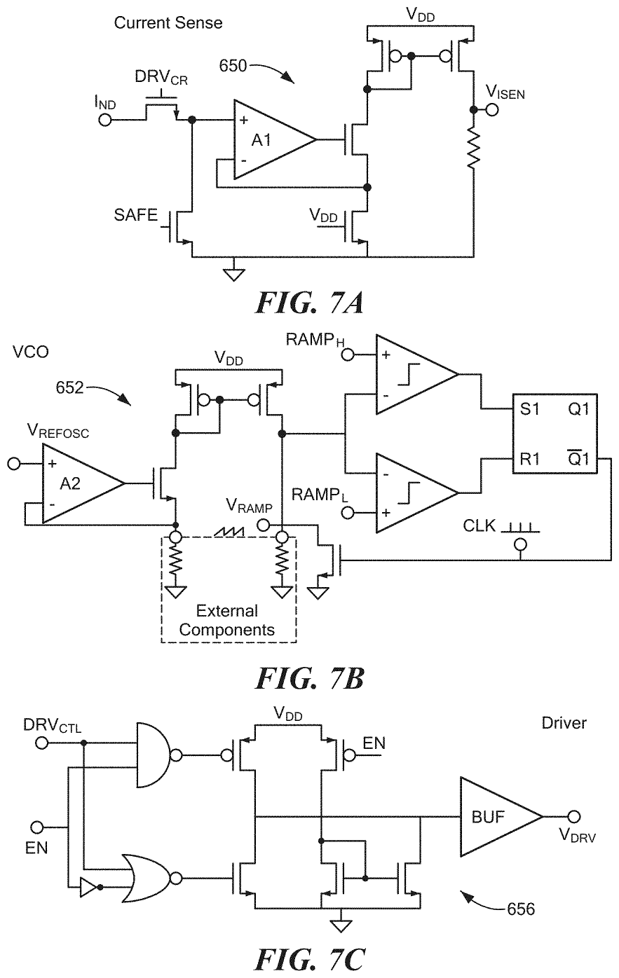

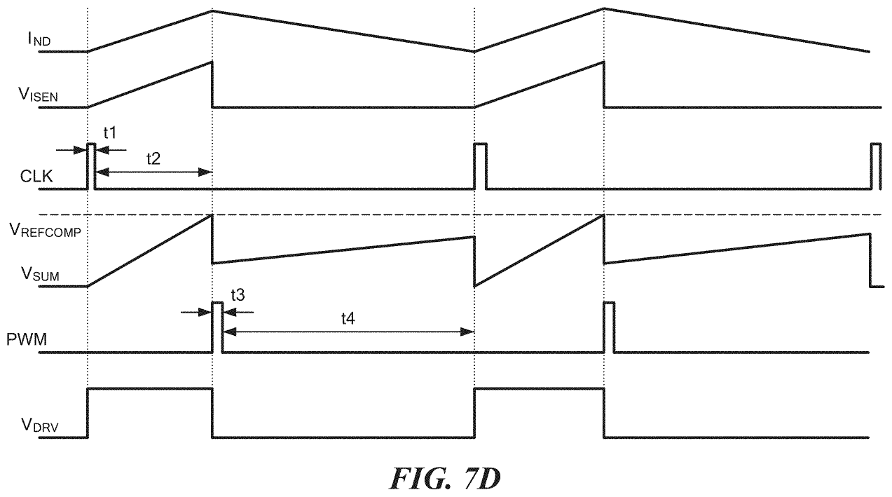

The LED driver 636 provides sufficient power to excite the blue LED with the proper intensity. It includes a current sensing block 650, a voltage-controlled oscillator (VCO) 652, a summer circuit, a comparator, an SR latch 654, and a driver 656, shown schematically in FIG. 7C. An off-chip inductor is employed to boost VDD to turn on the LED. The current sense block (FIG. 7A) measures the current (IIND) flowing through the inductor and outputs a signal (VISEN), which is then summed with the ramp signal (VRAMP) from the VCO, shown schematically in FIG. 7B, and compared with the current reference voltage VREFCOMP. This signal sets the limit of the maximum current in the inductor and keeps the driver stable. The timing diagram of the LED driver is illustrated in FIG. 7D.

The VCO generates the clock signal (CLK) and the ramp signal. VRAMP is summed with VISEN to create VSUM, which is then compared to an externally controlled reference voltage (VREFCOMP) in order to set the PWM signal. This signal resets the SR latch 654 and determines the pulse width of the DRVCTL signal. When EN is low, the driver is powered down, regardless of the DRVCTL. When enabled (EN=HIGH), VDRV controls the NMOS device based on the pulse-width modulated DRVCTL, and adjusts the current flowing through the LED. The EN signal is pulsed to reduce the power consumption of the readout. When EN is high, the LED driver pulls 16 mA current with VREFCOMP at 600 mV (increasing VREFCOMP increases IIND). When EN is low, the quiescent current of the LED driver 656, dominated by the current consumption of the VCO, is 180 mA. The externally controlled signals RAMPH, RAMPL, and VREFOSC, set the upper limit, the lower limit, and the frequency of the ramp waveform, respectively. Pulsation patterns of the blue light may vary, and are typically a series of rapid pulses interspersed between longer null intervals, as the oxygen diffusion has an inertial variance that can be effectively monitored periodically over 1-10 seconds to avoid excessive power drain.

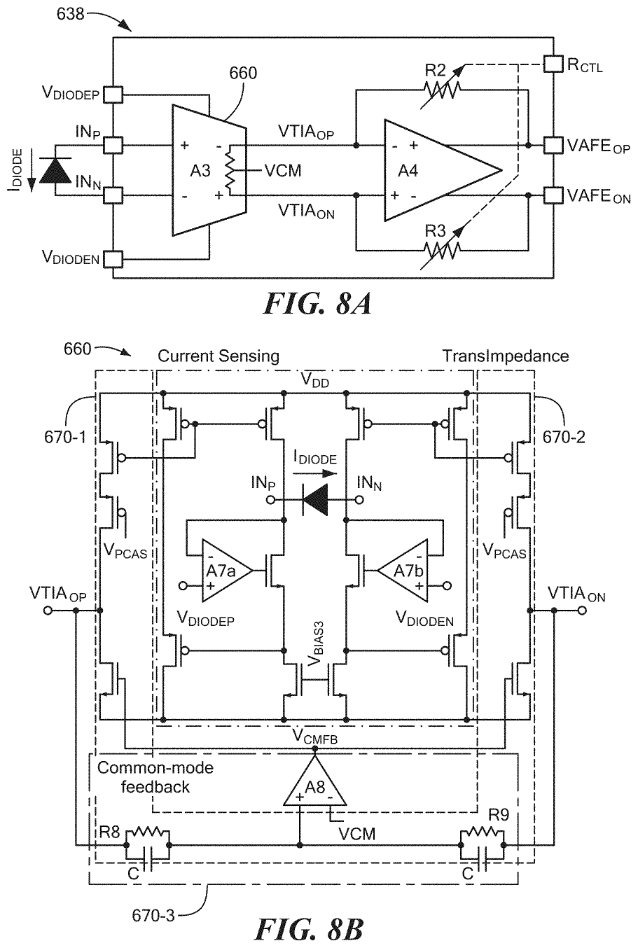

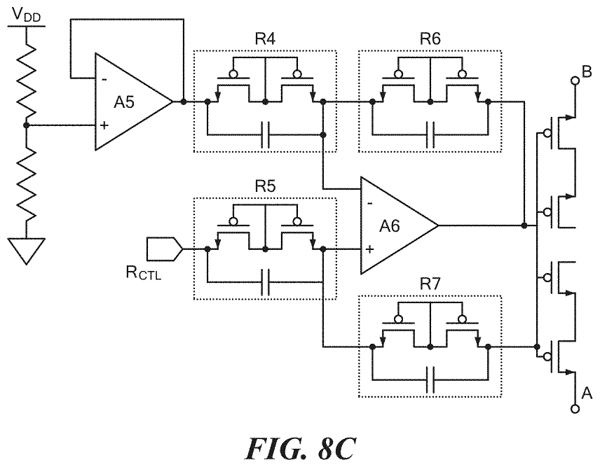

FIGS. 8A-8C shows a further detail of the analog front end 638 of FIG. 6. Referring to FIGS. 6-8C, in a particular arrangement, the AFE 638 includes two stages: a transimpedance amplifier (TIA) 660 and a variable gain amplifier 662 (VGA) 662. A differential TIA architecture minimizes the input impedance of the amplifier 662, which allows for small duty cycles and reduces common-mode noise. The cathode and the anode of the photodetector 212 connect to INP and INN inputs of the TIA 660, respectively.

The pulsing frequency of the system is based on the input impedance of the TIA and the capacitance of the photodetector 212. The TIA 660 may include current sensing 670-1, transimpedance 670-2, and common mode stages 670-3. The main purpose of the current-sensing stage 670-1 is to reduce the input impedance of the TIA with inner feedback loops created with amplifiers Ala and Alb and supplying a stable DC bias to the PD. The transimpedance stage 670-2 is designed to convert the current generated by the photodetector 212 to voltage with a gain of substantially around 50.1 kΩ using the feedback resistors R8 and R9 (shared with the common-mode feedback amplifier). The tunable gain is provided by the fully differential VGA 662, which follows the TIA. The variable gain is achieved by using pseudo resistors R2 and R3 controlled externally by RCTL.

FIG. 8C shows the schematic of the variable pseudoresistors. The design consists of a two-stage NMOS op-amp with indirect feedback compensation A5, a single-stage differential amplifier A6, and non-tunable pseudo-resistors, R4, R5, R6 and R7. Varying the control voltage RCTL changes the resistance value of tunable pseudo-resistors from a few hundred kΩ to several hundred Go.

The intervals of light and the wavelengths thereof are depicted above in an example arrangement. Other intensities, pulsing cycles, and wavelengths may be provided and/or varied to produce the described results, possibly with alternate granularity and precision. The logic 130 in the example circuits my be provided by any suitable logic circuit, integrated circuit and/or programmed set of instructions, executed by a processor and/or embodied in a hardware or software rendering in volatile or non-volatile memory.

FIG. 9 shows a relation between intensity and duration of remitted light. Referring to FIG. 9, and using oxygen as an example as shown herein, a graph 900 shows measured emitted light output for various O₂ concentrations 910 in the environment. As the partial pressure of oxygen increases (arrow 920), the output decreases generally faster, with variations on pressure. A resulting curve 930 approximates data points as the lifetime (duration) 932 of the emitted fluorescent lifetime decreases as partial pressure of oxygen (PO₂) 934 increases, as per a Stern-Volmer response. In other words, the quenching effect of oxygen reduces both the intensity (strength) and lifetime (duration) that the emitted red light is detected. The relation of intensity to duration allows measurement of either or both towards computing the PO₂, however measurement of lifetime tends to be more robust, as described above.

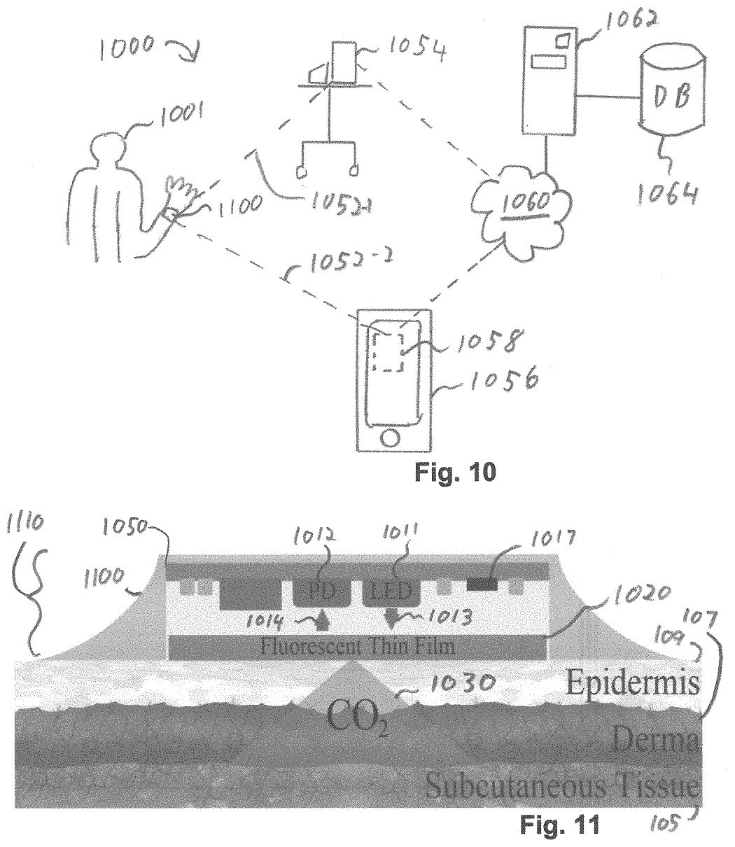

FIG. 10 shows a context diagram of an alternate configuration for gaseous monitoring and reporting. The disclosed approach is viable for a wearable device to measure a gaseous presence from transdermal diffusion of any suitable gas. For example, as with the oxygen measurement discussed above, a miniaturized, wireless luminescence-based continuous transcutaneous carbon dioxide monitoring wearable device overcomes the limitations of the traditional transcutaneous carbon dioxide monitors such as a need for a heating element and a large, expensive bedside monitor that prevent continuous monitoring outside a clinical setting. The measurement device employs an optical method, rather than an electrochemical method, and therefore does not require a heating element, thus allowing for wearable portability.

Configurations herein present a self-contained, wearable transcutaneous gaseous measurement device including a flexible, planar material having an emissive response based on transcutaneous carbon dioxide and a light source disposed for directing light at the flexible planar material. A sensor receives re-emitted light from the flexible planar material for indicating a concentration of CO₂ based on a partial pressure of the CO₂ (PtcCO₂), at the skin surface of the monitored patient. The sensor may be a photodiode responsive to an intensity of the re-emitted light, in which the intensity is indicative of a concentration of CO₂, and the photodiode has an output signal based on a concentration of the PtcCO₂. The light source may be an LED (Light Emitting Diode) with minimal power requirements, thus amenable to a portable (wearable) device. The LED emits a wavelength based on a sensitivity of the flexible planar material to CO₂. In the example configuration, the flexible planar material is an epidermal patch formed from a photoluminescent film having a sensitivity to a wavelength of light and the CO₂.

Referring to FIG. 10, in a patient monitoring environment 1000, a patient 1001 wears an epidermal, portable wireless transcutaneous sensing device 1100 (sensing device) on a wrist or other region suitable for receiving a strap or adhesive mounted fixture, such as an ankle, abdomen or chest. The device 1100 is operable with a transmission path 1052-1 . . . 1052-2 (1052 generally) to a patient monitor station 1054 or a more portable wireless device 1056. In a medical facility, a patient monitor station 1054 uses a wired or wireless transmission path 1052-1 to a rolled bedside cart, nurse station or similar, while a mobile configuration employs the transmission path 1052-2 to an application (app) 1058 on the wireless device 1056. In either case, the transmission path 1052 may continue to a public access network 1060 for transmission and storage of the gaseous measurements on a remote server 1062 and/or cloud storage 1064 facility.

The sensor patch device 1100 can therefore communicate via a wireless protocol to a base station which can either be medically monitored in a hospital setting or a smart device in a living environment. The data can be accessed in real-time. In addition to real time analysis, the data can also be securely stored. Post-processing of the data and customized machine learning algorithms can be applied to the stored data. This assists medical professionals for conducting detailed analysis for better informed medical decisions and tailored treatment plans for the patient. This system can lead to critical observations about response to therapies and natural resolutions of different conditions. It is also an accessible solution that can help provide more robust care options to patients in remote locations.

Patients with respiratory disorders, ranging from mild respiratory problems to more severe issues requiring mechanical ventilation constitute a major risk group which are inclined to suffer complete respiratory failure. In the case of a respiratory failure, the abnormal respiratory activity causes post-operative respiratory complications such as hypercapnia and hypocapnia, excessive and reduced carbon dioxide in the blood stream, respectively. Cardiac rhythm disorders, acute brain injury, and stroke are among the severe results of these complications. Failure of the respiration functions can also lead to diseases including chronic obstructive pulmonary disease (COPD), asthma, bronchitis, and respiratory distress syndrome (RDS). Respiratory diseases are the leading causes of death and disability, imposing enormous worldwide health burden. About one in twelve Americans have asthma, a lifelong disease, and the rate of asthma diagnoses increases every year. Adults with COPD and other respiratory issues constitute 4 to 12% of the population of the US. Therefore, adults with underlying respiratory diseases are another target group that may benefit from the disclosed approach.

FIG. 11 shows a schematic diagram of the configuration of FIG. 10 optimized for carbon dioxide detection. The above configuration, for oxygen detection, and the following configuration, amenable to CO₂ detection, are but an example arrangement of the sensing film responsive to a gaseous presence for emitting a particular sensory wavelength in response to a projected light of a predetermined wavelength.

Referring to FIGS. 10 and 11, an emitter 1011 is adapted to project light 1013 of a predetermined sensory wavelength, and a photoluminescent film 1020 opposed to the emitter receives the light from the emitter 1011 and is responsive to emit light 1014 of a sensed wavelength in response to the received light based on a gaseous exposure 1030 of the photoluminescent film 1020. An optical sensor 1012 is responsive to the sensed wavelength for returning a signal indicative of the sensed wavelength, and a correlation circuit 1050 for computing a gaseous presence based on the signal.

In a particular configuration, the sensor 1012 is a photodiode responsive to a fluorescence emission of the flexible planar material in a green light based on a blue light directed at the flexible planar material. The wearable epidermal patch connects to a correlation circuit for computing a PtcCO₂ from an inverse of an intensity of the fluorescence. A transmission interface 1017 coupled to the correlation circuit is adapted to receive a plurality of values based on the signal and transmit over the communication path 1052.

As with the O₂ configuration, the gaseous presence 1030 results from transcutaneous gases passing from the epidermal surface 109 to the photoluminescent film 1020. The photoluminescent film 1020, emitter 1011 and the optical sensor 1012 define a layered arrangement 1110 with the epidermal surface 109, such that the layered arrangement 1110 disposes the photoluminescent film 1020 opposed from the emitter 1011 and optical sensor 1012, and between the epidermal surface 109 and the correlation circuit 1050 having the emitter 1011 and optical sensor 1012.

The layered arrangement 1110 forms an generally flat or low-profile device 1100 such that the photoluminescent film 1020 is disposed in planar communication with the epidermal surface 109 and is responsive to transcutaneous gases forming the gaseous presence 1030 while passing from the epidermal surface 109 to the photoluminescent film 1020. The device 1100 arranges emitter and the optical sensor in a common plane on the circuit board and opposed to a plane defining the photoluminescent film, and in close proximity while sufficiently distal to allow the emitted light 1014 to reach the optical sensor or photodetector 1012.

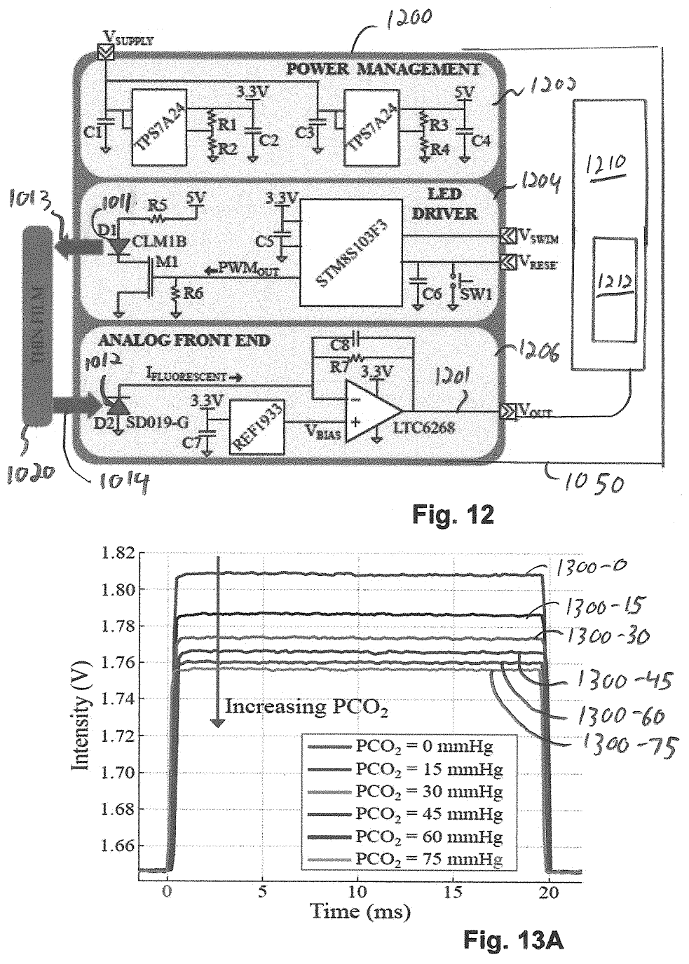

FIG. 12 shows a block diagram of the device in FIG. 10. The device 1100 includes a circuit board 1050 with a sensing circuit 1200. The sensing circuit orients the emitter 1011, optical sensor 1012 and correlation circuit 1210 in a planar arrangement adapted for engagement with the epidermal surface 109. The full device 1100 configuration takes the form wearable wireless sensor patch. Among the various vital parameters measurable through gaseous sensing, the device 1100 measures partial pressure of transcutaneous carbon dioxide (PtcCO₂).

The sensing system implemented by the circuit board 1050 allows for a low height profile, low power consumption, and wireless communication capability for extended, long-term wear and comfort. A particular benefit of the disclosed sensing system is that it is a wearable wireless optical-based system with no traditional heating element for transcutaneous carbon dioxide measurement. This grants the patient improved freedom of movement, better skin compliance, and doctors will receive more pertinent medical data to make better informed medical decisions and tailored treatment plans for the patient 1001.

Configurations of FIGS. 1-10 above demonstrate partial pressure of transcutaneous oxygen (PtcO₂) measurements based on an oxygen sensitive photoluminescent film. However, carbon dioxide sensitive photoluminescent films have not been utilized for PtcCO₂ monitoring. Configurations herein employ a photoluminescent film to sense PtcCO₂. The system measures PtcCO₂ using an optical method. The luminescent sensing film 1020 on the wearable patch device is placed on the patient's skin. The film is exposed to light from the light source such as a light-emitting diode (LED). The wavelength of the light will be dependent on the type of sensing film being used. The sensor patch can have one or more light sources. The intensity of re-emitted light from the film 1020 will be dependent on the concentration of the parameter (level of carbon dioxide) being measured. The change in the intensity of the re-emitted light is measured by the light detector such as a photodiode (PD). A system-on-chip (SoC) on the patch controls the functioning of the light source and measured the changes detected by the light detector.

Operational parameters of the circuit board 1050 of FIG. 12 provide that the sensed wavelength of emitted light 1014 is based on the predetermined sensory wavelength of light 1013 and a material sensitivity of the photoluminescent film 1020 to the gaseous environment. The device 1100 is a generally flat construction for nonobtrusive wearing and use. In a particular configuration, the planar arrangement 1110 has a thickness less than half of a shortest dimension of the circuit board 1050, and particular configuration exhibit a thickness less than 10% of the shortest dimension of the circuit board.

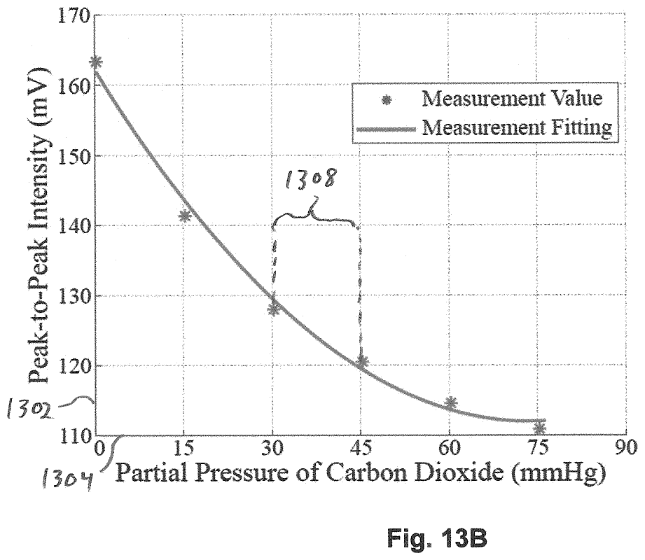

The photoluminescent film 1020 (film) is selected based on an ability to sense PtcCO₂. In the example configuration, the film 1020 emits green light (520 nm) when excited by a blue light (470 nm); this process is called fluorescence (or fluorescent emission). The fluorescence intensity is inversely proportional to the PtcCO₂. The circuit board 1050 measures the fluorescence intensity to determine the PtcCO₂. The schematic diagram of FIG. 12 shows a system architecture with three main blocks: the power management 1202, the LED driver 1204 for emitting blue light, and the analog front-end 1206 processing the signal 1201 indicative of the emitted green light intensity. FIGS. 13A and 13B show results of the device of FIGS. 10-12 for measuring transcutaneous carbon dioxide. In FIG. 13A, the signal 1201 from the photodetector 1012 is indicative of a fluorescence intensity of the sensed wavelength, and a correlation circuit 1210 includes concentration logic 1212 for computing a concentration of a predetermined substance defined by the gaseous presence. Intensity 1300-0 . . . 1300-75 of the luminescent emission is measured by the output of the PtcCO₂ monitor for different values of partial pressure of carbon dioxide. Intensity plot 1300-0 shows an intensity reading of around 1.81 V at 0 mmHg, incrementing in 15 mm Hg increments to an intensity of around 1.755 V at 75 mmHG. FIG. 13B shows a result from the concentration logic 1212 where the fluorescence intensity 1302 is inversely proportional to a partial pressure 1304 of carbon dioxide in the gaseous presence, and the concentration logic 1212 computes a carbon dioxide concentration. FIG. 13B demonstrates the fluorescence intensity measured with the prototype monitor for different partial pressure values of carbon dioxide levels ranging from 0 mmHg to 75 mmHg. The results illustrate that the fluorescence intensity is inversely proportional to the partial pressure of carbon dioxide. As shown in FIG. 13B, the measurement range 1308 of the monitor is within the clinically relevant range for healthy humans, 35-45 mmHg. This measurement demonstrates of detection of the CO₂ concentration.

Conventional approaches to transcutaneous gaseous measurement include the following. U.S. Pat. No. 4,930,506 shows a Combined Sensor for the Transcutaneous Measurement of Oxygen and Carbon Dioxide in the Blood.

The combined sensor with a thermostatic heating device for the simultaneous and continuous transcutaneous measurement of oxygen and of carbon dioxide operates on the principle of pH measurement in an electrolyte, and which is separated from the measuring site on the skin by a membrane which is permeable to both carbon dioxide and light. An advantage of the disclosed approach is that it can be operated at a low operating temperature of, for example, 42° C. or less, so that continuous measurement for 24 hours, for example, is possible without resetting or recalibrating the sensor, and thus it is simple to use in the domestic milieu. In contrast, the disclosed approach detects carbon dioxide with an optical method while this device uses the principle of pH measurement in an electrolyte, relying on an electrochemical method. This device therefore requires a heating element.

US 2019/0021672, by Bremer, suggests a method of glucose analysis based on percutaneous (i.e. skin piercing) and utilizes a hemoglobin polymer matrix embedded within siloxane. There is no showing, teaching or disclosure of an epidermally sensed gaseous response. The disclosed approach is for skin diffused CO₂, not blood testing for glucose.

US 2010/0130842 shows a Device and Method for Transcutaneous Determination of Blood Gases. The '842 device for the transcutaneous determination of blood gases includes an electrochemical measuring device for the measurement of the PtcCO₂. The electrochemical measuring device is composed of a micro-pH electrode and an Ag/AgCl reference electrode. A change in the carbon dioxide value at the skin surface creates a pH change in an electrolyte solution where the micro-pH and Ag/AgCl reference electrodes reside. The pH in the electrolyte changes proportional to the carbon dioxide concentration. A heating system is used together with the electrochemical measuring device for heating up the skin to 40-44° C. in order to increase carbon dioxide diffusion. The disclosed approach uses an optical method, rather than an electrochemical method, and thus does not require a heating element, allowing for a wearable and wireless implementation.

A Sentec® device RD-007429 defines a commercial monitor, which uses a chemical electrode to measure transcutaneous carbon dioxide using a wired patient interface, and requires a bulky bedside monitor.

U.S. Pat. No. 8,771,184 suggests Wireless Medical Diagnosis and Monitoring Equipment via a method of medical diagnosis and monitoring using equipment that has wireless electrodes, which are attached to the surface of the skin of the patient. The method comprises collection of data from a patient, converting the data to digital form and transmitting the digital data wirelessly from the electrodes attached to the patient's body to a base station located away from the patient. This device uses the conventional electrochemical method for measuring PtcCO₂ which requires a heating element.

In U.S. Pat. No. 10,307,090, a Sensor for Detection of Gas and Method for Detection of Gas suggests an infrared-based sensor for detection of gas, in particular for detection of carbon dioxide. The sensor has a contact face which can be directed towards a measuring site. The sensor includes at least one radiation source, a measurement volume for receiving the gas to be measured, and at least a first detector for detection of radiation transmitted from the source to the first detector through the measurement volume. The sensor has a path of the radiation between source and first detector. The radiation propagates along the path in a non-imaging way. The '090 approach is based on an infrared sensing principle. No actual electronic interface is presented.

In Tipparaju et al. “Wearable Transcutaneous CO₂ Monitor Based on Miniaturized Nondispersive Infrared Sensor”, IEEE Sensors, 2021, the research reports the development of a truly wearable sensor for continuous monitoring of transcutaneous carbon dioxide using miniaturized nondispersive infrared sensor augmented by hydrophobic membrane to address the humidity interference. This system, however, is based on an infrared sensing principle.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An epidermal, portable wireless transcutaneous sensing device, comprising:
an emitter adapted to project light of a predetermined sensory wavelength;
a photoluminescent film opposed to the emitter for receiving the light from the emitter and responsive to emit light of a sensed wavelength in response to the received light based on a gaseous exposure of the photoluminescent film;
an optical sensor responsive to the sensed wavelength for returning a signal indicative of the sensed wavelength; and
a correlation circuit for computing a gaseous presence based on the signal.

2. The device of claim 1 wherein the emitter and the optical sensor are disposed in a common plane and opposed to a plane defining the photoluminescent film.

3. The device of claim 1 further comprising a circuit board including a sensing circuit, the sensing circuit orienting the emitter, optical sensor and correlation circuit in a planar arrangement adapted for engagement with an epidermal surface.

4. The device of claim 1 wherein the photoluminescent film, emitter and the optical sensor define a layered arrangement with an epidermal surface, the layered arrangement disposing the photoluminescent film opposed from the emitter and optical sensor, and between an epidermal surface and the emitter and optical sensor.

5. The device of claim 1 wherein the photoluminescent film is disposed in planar communication with an epidermal surface and responsive to transcutaneous gases passing from the epidermal surface to the photoluminescent film.

6. The device of claim 1 wherein the signal is indicative of a fluorescence intensity of the sensed wavelength, and the correlation circuit includes concentration logic for computing a concentration of a predetermined substance defined by the gaseous presence.

7. The device of claim 6 wherein the fluorescence intensity is inversely proportional to a partial pressure of carbon dioxide in the gaseous presence, and the concentration logic computes a carbon dioxide concentration.

8. The device of claim 5 wherein the gaseous presence results from transcutaneous gases passing from the epidermal surface to the photoluminescent film.

9. The device of claim 6 further comprising a transmission interface coupled to the correlation circuit and adapted to receive a plurality of values based on an iteration of the computed concentration.

10. The device of claim 1 wherein the sensed wavelength is based on the predetermined sensory wavelength and a material sensitivity of the photoluminescent film to the gaseous environment.

11. The device of claim 3 wherein the planar arrangement has a thickness less than half of a shortest dimension of the circuit board.

12. The device of claim 3 wherein the planar arrangement has a thickness less than 10% of the shortest dimension of the circuit board.

13. A system for portable, epidermal wireless transcutaneous sensing, comprising:
an emitter adapted to project light of a predetermined sensory wavelength;
a photoluminescent film opposed to the emitter for receiving the light from the emitter and responsive to emit light of a sensed wavelength in response to the received light based on a gaseous exposure of the photoluminescent film;
an optical sensor responsive to the sensed wavelength for returning a signal indicative of the sensed wavelength;
a correlation circuit for computing a gaseous presence based on the signal; and
a transmission path for transmitting a value of the computed gaseous presence.
